SEQUENCE LISTING (22 sequences)

SEQ ID NO: 1 - protein, 451 aa, Artificial sequence
  Artificial polypeptide
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 213 aa, Artificial sequence
  Artificial polypeptide
DIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 451 aa, Artificial sequence
  Artificial polypeptide
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4-6 - skipped (3 entries)

SEQ ID NO: 7 - protein, 4 aa, Artificial sequence
  Artificial oligopeptide
GGSG

SEQ ID NO: 8 - protein, 8 aa, Artificial sequence
  Artificial oligopeptide
GGSGGGSG

SEQ ID NO: 9 - protein, 9 aa, Artificial sequence
  Artificial oligopeptide
SGGSGGSGG

SEQ ID NO: 10 - protein, 12 aa, Artificial sequence
  Artificial oligopeptide
GGSGGGSGGGSG

SEQ ID NO: 11 - protein, 15 aa, Artificial sequence
  Artificial oligopeptide
GGGGSGGGGSGGGGS

SEQ ID NO: 12 - protein, 6 aa, Artificial sequence
  Artificial oligopeptide
GSGSGS

SEQ ID NO: 13 - protein, 18 aa, Artificial sequence
  Artificial oligopeptide
EGRGSLLTCGDVEENPGP

SEQ ID NO: 14 - protein, 19 aa, Artificial sequence
  Artificial oligopeptide
ATNFSLLKQAGDVEENPGP

SEQ ID NO: 15 - protein, 20 aa, Artificial sequence
  Artificial oligopeptide
QCTNYALLKLAGDVESNPGP

SEQ ID NO: 16 - protein, 22 aa, Artificial sequence
  Artificial oligopeptide
VKQTLNFDLLKLAGDVESNPGP

SEQ ID NO: 17 - protein, 9 aa, Artificial sequence
  Artificial oligopeptide
FTFSHYIMM

SEQ ID NO: 18 - protein, 21 aa, Artificial sequence
  Artificial oligopeptide
GIYSSGGITVYADSVKGRFTI

SEQ ID NO: 19 - protein, 13 aa, Artificial sequence
  Artificial oligopeptide
RRIGVPRRDEFDI

SEQ ID NO: 20 - protein, 11 aa, Artificial sequence
  Artificial oligopeptide
RASQSISSWLA

SEQ ID NO: 21 - protein, 14 aa, Artificial sequence
  Artificial oligopeptide
YKASTLESGVPSRF

SEQ ID NO: 22 - protein, 8 aa, Artificial sequence
  Artificial oligopeptide
QQYNTYWT

(19) United States
(12) Patent Application Publication
(10) Pub. No.: US 2023/0038502 A1
(43) Pub. Date: Feb. 9, 2023

(54) ADENO ASSOCIATED VIRAL VECTOR DELIVERY OF ANTIBODIES FOR THE TREATMENT OF DISEASE MEDIATED BY DYSREGULATED PLASMA KALLIKREIN

(71) Applicant: Shire Human Genetic Therapies, Inc., Lexington, MA (US)

(72) Inventors: Jon Kenniston, Lexington, MA (US); Florie Borel, Lexington, MA (US); Madhusudan Natarajan, Lexington, MA (US); Vivian Choi, Lexington, MA (US); Dan Sexton, Lexington, MA (US); Alexey Seregin, Lexington, MA (US)

(21) Appl. No.: 17/618,193

(22) PCT Filed: Jun. 11, 2020
(86) PCT No.: PCT/US2020/037189

§ 371 Date: Dec. 10, 2021

Related U.S. Application Data
(60) Provisional application No. 62/860,101, filed on Jun. 11, 2019.

(51) Int. Cl. C12N 15/86; A61K 48/00; C07K 16/40

(52) U.S. Cl. CPC C12N 15/86; A61K 48/0058; C07K 16/40; C12N 2750/14143; C12N 2830/008; C12N 2830/48; C12N 2840/203

(57) ABSTRACT

The present disclosure provides, among other things, a recombinant adeno-associated viral (rAAV) vector encoding an agent that inhibits the proteolytic activity of plasma kallikrein. The disclosure also provides, a recombinant adeno-associated viral (rAAV) vector encoding an anti/plasma kallikrein antibody heavy drain and an anti-plasma kallikrein antibody light chain.

### CROSS-REFERENCE TO RELATED APPLICATIONS

This application is a 35 U.S.C. § 371 National Stage Application of International Application No. PCT/US20/37189, filed on Jun. 11, 2020, which claims benefit of, and priority to, U.S. Ser. No. 62/860,101 filed on Jun. 11, 2019, the content of which are incorporated herein in their entireties.

### INCORPORATION BY REFERENCE OF SEQUENCE LISTING

The content of the text file named “SHR-2008US1_sequence_ST25.txt”, which was created on Mar. 15, 2022 and is 14 pkilobytes in size, is hereby incorporated by reference in its entirety.

### BACKGROUND

Dysregulated plasma kallikrein activity may lead to excess production of the proinflammatory and vasoactive peptide, bradykinin. An example of such a disease is hereditary angioedema (HAE), a rare, but potentially life-threatening disorder characterized by unpredictable and recurrent attacks of vasodilation manifesting as subcutaneous and submucosal angioedema. In some cases, HAE is associated with low plasma levels of C1-inhibitor (type I), while in other cases the protein circulates in normal or elevated amounts but it is dysfunctional (type II). C1 inhibitor is the main regulator of plasma kallikrein activity. Symptoms of HAE attacks include swelling of the face, mouth and/or airway that occur spontaneously or are triggered by mild trauma. Edematous attacks affecting the airways can be fatal. In addition to acute inflammatory flares, excess plasma kallikrein activity has also been associated with chronic conditions, such as autoimmune diseases, including lupus erythematosus.

Various strategies for the treatment of C1-INH deficiencies or dysfunctions have been contemplated and developed, including for example inhibiting members of the contact system. For example, lanadelumab is a fully human monoclonal antibody inhibitor of plasma kallikrein that has been approved for the treatment of HAE.

Use of vectors that produce proteins, including antibodies, in vivo is desirable for the treatment of disease, but is limited by various factors including poor antibody production following delivery to a subject.

### SUMMARY OF THE INVENTION

The present invention provides efficient and robust recombinant adeno-associated viral (rAAV) vectors that encode anti-plasma kallikrein antibodies. The present invention is, in part, based on the surprising discovery that specific, recombinant AAV vectors that encode an anti-plasma kallikrein antibody heavy chain and an anti-plasma kallikrein antibody light chain results in the in vivo production of high levels of functional anti-plasma kallikrein antibodies. Specifically, the rAAV leads to robust and sustained production of anti-plasma kallikrein mAbs in vivo and the vector-mediated expressed anti-plasma kallikrein antibodies retain targeting activity equivalent to antibody protein produced by traditional recombinant expression methods (e.g., CHO cells). Prior to the present invention, delivery of anti-plasma kallikrein antibodies through the administration of rAAV vectors carrying a desired payload resulted in unknown quantities of active antibody production. Therefore, prior to the present invention it was not predictable or feasible to use rAAV vectors encoding anti-plasma kallikrein for the treatment of C1-INH deficiencies or disorders, including for example, hereditary angioedema.

In some aspects, provided herein is a recombinant adeno-associated viral (rAAV) vector encoding a full length antibody comprising an anti-plasma kallikrein antibody heavy chain and an anti-plasma kallikrein antibody light chain.

In some embodiments, the anti-plasma kallikrein antibody heavy chain and the anti-plasma kallikrein antibody light chain are linked via a linker.

In some embodiments, the linker comprises a cleavable linker.

In some embodiments, the linker comprises a non-cleavable linker.

In some embodiments, the anti-plasma kallikrein antibody heavy chain and the anti-plasma kallikrein antibody light chain are controlled by a single promoter.

In some embodiments, the anti-plasma kallikrein antibody heavy chain and the anti-plasma kallikrein antibody light chain are controlled by separate promoters.

In some embodiments, the single promoter or one or more of the separate promoters is selected from a ubiquitous promoter, a tissue-specific promoter, or a regulatable promoter.

In some embodiments, the tissue-specific promoter is a liver-specific promoter.

In some embodiments, the liver-specific promoter comprises a promoter selected from human transthyretin promoter (TTR), modified hTTR (hTTR mod.), α-Antitrypsin promoter, Liver Promoter 1 (LP1), TRM promoter, human factor IX pro/liver transcription factor-responsive oligomers, LSP, CMV/CBA promoter (1.1 kb), CAG promoter (1.7 kb), mTTR, modified mTTR, mTTR pro, mTTR enhancer, or the basic albumin promoter.

In some embodiments, the liver-specific promoter is human transthyretin promoter (TTR).

In some embodiments, the regulatable promoter is an inducible or repressible promoter.

In some embodiments, the vector further comprises one or more of the following: a 5′ and a 3′ inverted terminal repeat, an intron upstream of the sequence, and a cis-acting regulatory module (CRM).

In some embodiments, the vector further comprises a Woodchuck Posttranscriptional Regulatory Element (WPRE) sequence.

In some embodiments, the WPRE sequence is modified.

In some embodiments, WPRE contains a mut6delATG modification. In some embodiments, the WPRE is a WPRE3 variant.

In some embodiments, the CRM is liver-specific CRM.

In some embodiments, the CRM is CRM8.

In some embodiments, the vector comprises at least three CRMs.

In some embodiments, the vector comprises three CRM8.

In some embodiments, the rAAV vector comprises an internal ribosome entry site (IRES) sequence.

In some embodiments, the anti-plasma kallikrein antibody light chain and/or heavy chain comprise one or more mutations that enhance the half-life and/or reduce the effector function of the antibody.

In some embodiments, the one or more mutations comprise LALA mutations (L234A and L235A) and/or NHance mutations (H433K and N434F).

In some embodiments, the one or more mutations comprise LALA mutations (L234A and L235A).

In some embodiments, the AAV vector is selected from AAV1, AAV2, AAV3, AAV4, AAV5, AAV6, AAV7, AAV8, AAV9, AAV10, AAV11, or AAVrh.10.

In some embodiments, the rAAV vector capsid is engineered.

In some embodiments, the engineered rAAV vector comprises an AAV capsid sequence with a modified amino acid sequence.

In some embodiments, the modified amino acid sequence comprises insertion, deletion or substitution of one or more amino acid residues.

In some embodiments, the rAAV capsid is naturally derived.

In some embodiments, the rAAV vector capsid is AAV8.

In some embodiments, the cleavable sequence is a furin cleavable sequence.

In some embodiments, the furin cleavable sequence is followed by a linker and a 2A sequence.

In some embodiments, the linker is a GSG linker.

In some embodiments, the 2A sequence is a T2A, P2A, E2A or an F2A sequence.

In some embodiments, the 2A sequence is a P2A sequence.

In some embodiments, the vector further encodes a secretion signal.

In some embodiments, the secretion signal is a naturally-occurring signal peptide.

In some embodiments, the secretion signal is an artificial signal peptide.

In some embodiments, the anti-plasma kallikrein antibody heavy chain and the anti-plasma kallikrein antibody light chain produce a functional anti-plasma kallikrein antibody capable of binding to plasma kallikrein.

In some embodiments, the anti-plasma kallikrein antibody inhibits the proteolytic activity of plasma kallikrein.

In some embodiments, the antibody binds to the plasma kallikrein active site.

In some embodiments, the binding occludes the active site of plasma kallikrein.

In some embodiments, the binding inhibits the activity of plasma kallikrein.

In some embodiments, the antibody does not bind prekallikrein.

In some embodiments, the anti-plasma kallikrein antibody heavy chain and the anti-plasma kallikrein antibody light chain are expressed from the same vectors.

In some embodiments, the anti-plasma kallikrein antibody heavy chain and the anti-plasma kallikrein antibody light chain are expressed from distinct rAAV vectors.

In some embodiments, the anti-plasma kallikrein antibody heavy chain and the anti-plasma kallikrein antibody light chain are expressed from separate rAAV vectors.

In some embodiments, the vector further comprises a 5′ and a 3′ inverted terminal repeat (ITR), one or more enhancer elements, and/or a poly(A) tail.

In some embodiments, the one or more enhancer elements are selected from clusters of transcription factor binding sites and/or WPRE sequences.

In some aspects, a recombinant adeno-associated virus (rAAV) comprising an AAV8 capsid and an rAAV vector is provided, said vector comprising: (a) a 5′ inverted terminal repeat (ITR); (b) a cis-acting regulatory module (CRM); (c) a liver specific promoter; (d) an anti-plasma kallikrein antibody heavy chain sequence and an anti-plasma kallikrein antibody light chain sequence; (e) a woodchuck hepatitis virus posttranscriptional regulatory element (WPRE); and (f) a 3′ ITR.

In some embodiments, the liver specific promoter comprises a promoter selected from human transthyretin promoter (TTR), modified hTTR (hTTR mod.), α-Antitrypsin promoter, Liver Promoter 1 (LP1), TRM promoter, human factor IX pro/liver transcription factor-responsive oligomers, LSP, CMV/CBA promoter (1.1 kb), CAG promoter (1.7 kb), mTTR, modified mTTR, mTTR pro, mTTR enhancer, or the basic albumin promoter.

In some embodiments, the liver specific promoter comprises the human transthyretin promoter.

In some embodiments, the CRM is a liver specific CRM.

In some embodiments, the vector comprises at least CRMs.

In some embodiments, the vector comprises three CRM8.

In some embodiments, the WPRE sequence is modified.

In some embodiments, the WPRE sequence is WPRE mut6delATG.

In some aspects, a method of treating a disease or disorder associated with a deficiency or dysregulation in the activated kallikrein-kinin pathway in a subject in need thereof is provided, said method comprising administering a recombinant adeno-associated viral vector (rAAV) as described herein.

In some embodiments, the deficiency or dysregulation in the activated kallikrein-kinin pathway is a disease or disorder associated with a deficiency in C1 esterase inhibitor.

In some embodiments, the rAAV vector is administered by intravenous, subcutaneous, or transdermal administration.

In some embodiments, the transdermal administration is by gene gun.

In some embodiments, the disorder associated with a deficiency or dysregulation in the activated kallikrein-kinin pathway or a deficiency in C1 esterase inhibitor is hereditary angioedema (HAE), acquired angioedema (AAE), angioedema with normal C1 inhibitor, diabetic macular edema, migraine, oncology, neurodegenerative diseases, Alzheimer's disease, rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, cerebral edema, pulmonary embolism, stroke, clotting induced by ventricular assistance devices or stents, head trauma or peri-tumor brain edema, sepsis, acute middle cerebral artery (MCA) ischemic event, restenosis, systemic lupus erythematosis nephritis/vasculitis, or burn injury.

In some embodiments, the disorder associated with a deficiency in C1 esterase inhibitor is HAE. In some embodiments, the disorder is acquired angioedema (AAE). In some embodiments, the disorder is angioedema with normal C1 inhibitor. In some embodiments, the disorder is diabetic macular edema. In some embodiments, the disorder is migraine. In some embodiments, the disorder is a cancer. In some embodiments, the disorder is a neurodegenerative disease. In some embodiments, the disorder is Alzheimer's disease. In some embodiments, the disorder is rheumatoid arthritis. In some embodiments, the disorder is gout. In some embodiments, the disorder is intestinal bowl disease. In some embodiments, the disorder is oral mucositis. In some embodiments, the disorder is neuropathic pain. In some embodiments, the disorder is inflammatory pain. In some embodiments, the disorder is spinal stenosis-degenerative spine disease. In some embodiments, the disorder is arterial or venous thrombosis. In some embodiments, the disorder is post-operative ileus. In some embodiments, the disorder is aortic aneurysm. In some embodiments, the disorder is osteoarthritis. In some embodiments, the disorder is vasculitis. In some embodiments, the disorder is edema. In some embodiments, the disorder is cerebral edema. In some embodiments, the disorder is pulmonary embolism. In some embodiments, the disorder is stroke. In some embodiments, the disorder is clotting induced by ventricular assistance devices or stents. In some embodiments, the disorder is head trauma or peri-tumor brain edema. In some embodiments, the disorder is sepsis. In some embodiments, the disorder is acute middle cerebral artery (MCA) ischemic event. In some embodiments, the disorder is restenosis. In some embodiments, the disorder is systemic lupus erythematosis nephritis/vasculitis. In some embodiments, the disorder is burn injury.

In some embodiments, the HAE is type I, II, or III.

In some embodiments, the rAAV vector is episomal following administration.

In some embodiments, following administration the anti-plasma kallikrein antibody heavy chain and light chain assemble into a functional antibody.

In some embodiments, the antibody is IgG.

In some embodiments, the functional anti-plasma kallikrein antibody is detectable in plasma of the subject at about 2 to 6 weeks post administration of the rAAV vector.

In some embodiments, the functional anti-plasma kallikrein antibody is detectable in plasma of the subject at about 4 weeks post administration of the rAAV vector.

In some embodiments, the anti-plasma kallikrein antibody heavy chain comprises a CDR1 comprising an amino acid sequence of SEQ ID NO: 17, a CDR2 comprising an amino acid sequence of SEQ ID NO: 18, and a CDR3 comprising an amino acid sequence of SEQ ID NO: 19. In some embodiments, the anti-plasma kallikrein antibody light chain comprises a CDR1 comprising an amino acid sequence of SEQ ID NO: 20, a CDR2 comprising an amino acid sequence of SEQ ID NO: 21, and a CDR3 comprising an amino acid sequence of SEQ ID NO: 22.

In some embodiments, the anti-plasma kallikrein antibody heavy chain has at least about 50%, 55%, 60%, 65%, 70%, 75%, 80%, 85%, 90%, 95% or more sequence identity with SEQ ID NO: 1.

In some embodiments, the anti-plasma kallikrein antibody heavy chain is identical to SEQ ID NO: 1.

In some embodiments, the anti-plasma kallikrein antibody light chain has at least about 50%, 55%, 60%, 65%, 70%, 75%, 80%, 85%, 90%, 95% or more sequence identity with SEQ ID NO: 2.

In some embodiments, the anti-plasma kallikrein antibody light chain is identical to SEQ ID NO: 2.

In some embodiments, the anti-plasma kallikrein antibody light chain and/or heavy chain comprise one or more mutations that enhance the half-life and/or reduce the effector function of the antibody.

In some embodiments, the anti-plasma kallikrein antibody heavy chain has at least about 80%, 85%, 90%, 95% or more sequence identity with SEQ ID NO: 3.

In some embodiments, the anti-plasma kallikrein antibody heavy chain has an amino acid sequence of SEQ ID NO: 3.

### BRIEF DESCRIPTION OF THE DRAWINGS

FIG. 1 are schematic diagrams that illustrate an exemplary gene therapy approach using an rAAV vector encoding an anti-plasma kallikrein antibody. FIG. 1 depicts an AAV vector encoding anti-plasma kallikrein antibody administered intravenously (IV) to a subject in need; the vector is translated into functional anti-plasma kallikrein antibody, which is secreted into the circulation of the subject; and the antibody results in binding and inhibition of plasma kallikrein in the subject. IV=intravenous; HC=heavy chain; LC=light chain.

FIG. 2A is a series of schematics that show the rAAV constructs (from top to bottom of the schematic series) 1) an IgG (−) control construct; 2) an anti-PKa IgG+LALA construct; 3) an anti-PKa Fab construct; and 4) an anti-PKa IgG construct. FIG. 2B is a schematic that shows a rAAV construct comprising liver-specific promoter and/or enhancer elements, a WPRE element, and human secretion signals.

FIG. 3A, FIG. 3B, and FIG. 3C are graphs that show the total IgG and the active IgG levels in mouse plasma respectively at 2 and 4 weeks after IV administration of the indicated vector. Total IgG was quantified with an ELISA utilizing an anti-Fc antibody detection system, while the active IgG ELISA quantified only expressed IgGs that bound active PKa. Samples from left to right for each of the graphs correspond to the following treatments: null vector control (no transgene expression); control vector comprising coding sequence of an unrelated control antibody; vector comprising coding sequence of the anti-PKa antibody with LALA mutation; vector comprising coding sequence of the anti-PKa antibody Fab domains; vector comprising coding sequence of the full length anti-PKa antibody. The data presented on the graph for these samples were obtained 2 weeks and 4 weeks post-dose. The rightmost condition corresponds to the level of anti-PKa IgG (with LALA mutation) that was injected as a protein sample 2 hours prior to plasma collection. FIG. 3C is the same graph as shown in FIG. 3A with the addition of overlain horizontal lines which show therapeutic IgG level, and the range exceeding the therapeutic level, for treatment of HAE. BLQ=below quantification. FIG. 3D is a graph that shows the total IgG and the active IgG levels in mouse plasma at 28 days after intravenous administration of vectors—vehicle, rAAV8-1, rAAV8-2, and rAAV8-3. Vectors rAAV8-1, rAAV8-2, and rAAV8-3 are primarily derived from a rAAV construct shown in FIG. 3E with some variations in terms of promoter and/or enhancer elements, secretion signal, and WPRE element. Vector rAAV8-1 includes a CB promoter and a murine secretion signals, but no WPRE element. Vector rAAV8-2 includes a hTTR+3xCRM8 promoter and murine secretion signals, but no WPRE element. Vector rAAV8-3 includes a hTTR+3xCRM8 promoter, a human secretion signals, and a WPRE mut6 element. FIG. 3F is a graph that shows the active IgG levels in mouse plasma over 16 weeks after intravenous administration of rAAV8-2 vector at a 1×10¹¹ vg/kg dose in mice (n=8).

FIG. 4 is an image of a representative western blot for detection of heavy and light chains of the in vivo-expressed antibody from the plasma of mice treated with rAAV8-PKa IgG; LALA. Samples were collected at Day 28 after administration of the indicated vectors. For comparison, purified anti-PKa mAb (expressed and purified from traditional plasmid transfection of CHO cells) is included on the blot.

FIG. 5A is a schematic diagram that depicts an assay for measuring ex vivo bioactivity of the rAAV vector derived antibodies. The assay measures plasma kallikrein (“PKa”) activity using a fluorogenic peptide substrate in plasma samples obtained before and after administration of the respective vectors. FIG. 5B is a graph showing the ex vivo bioactivity of plasma collected at 14 and 28 days after respective antibody administration as indicated (or 0 and 2 hrs post IV injection of the protein IgG control). The bioactivity was measured as percent inhibition of plasma kallikrein activity.

FIG. 6 show a series of photomicrographs of representative immunohistochemistry of mouse liver sections after administration of the following: rAAV8 anti-plasma kallikrein antibody with LALA mutation (“rAAV8 PKa IgG LALA”) (left); rAAV8 null vector control (middle); and uninjected control (right). Arrows indicate positive staining for the specific antibody.

FIG. 7A, FIG. 7B and FIG. 7C show varying magnifications of a series of photomicrographs of representative immunohistochemistry of mouse liver sections 4 weeks after injection of the rAAV vector comprising coding sequence of full length IgG or the Fab, as indicated. Positive staining of hepatocytes and sinusoid cells are indicated by wide arrows and thin arrows respectively in FIG. 7A.

FIG. 8 is a graph that shows percent active anti-PKa IgG LALA from mice injected with rAAV8 anti-PKa IgG LALA vectors and assessed after 2 weeks and 4 weeks following administration. The rightmost condition is an IV injection of purified anti-PKa LALA antibody followed by assessment of percent active anti-PKA IgG at a pre-dose period (time zero) and 2 hours after administration.

FIG. 9 shows mass spectra measuring the molecular weight of intact and processed antibodies. Graphs at the left panel of this figure represent purified/standard antibody. Graphs at the right panel of this figure represent the anti-PKa antibody produced in rAAV8-treated mouse plasma. Intact antibody refers to the native antibody that is produced in the rAAV8-treated mouse plasma. The purified anti-PKa mAb was spiked in a blank mouse plasma to create a purified intact sample. The processed antibody refers to the anti-PKa antibody that has undergone reduction, deglycosylation, or deglycosylation and reduction both.

FIG. 10 is a graph showing the ex vivo bioactivity of anti-PKa antibody produced in a rAAV8-treated mouse plasma sample collected at 28 days after rAAV8 construct intravenous administration. The potency of the anti-PKa antibody produced in the rAAV8-treated mouse plasma in inhibiting the kallikrein-kinin pathway was compared with the potency of a commercially available inhibitor, Takhzyro™ (lanadelumab, a fully human monoclonal antibody inhibitor of plasma kallikrein), in inhibiting the same pathway. The bioactivity was measured in terms of percent inhibition of plasma kallikrein activity as a function of anti-PKa antibody concentration.

### DEFINITIONS

In order for the present invention to be more readily understood, certain terms are first defined below. Additional definitions for the following terms and other terms are set forth throughout the specification.

As used in the specification and claims, the singular form “a”, “an” and “the” include plural references unless the context clearly dictates otherwise. For example, the term “a cell” includes a plurality of cells, including mixtures thereof.

2A sequence: As used herein “2A” or “2A sequence” or “2A peptide” refers to a class of self-cleavable peptides. Example of 2A peptides include T2A, P2A, E2A, and F2A. T2A has a sequence of EGRGSLLTCGDVEENPGP (SEQ ID NO: 13); P2A has a sequence of ATNFSLLKQAGDVEENPGP (SEQ ID NO: 14); E2A has a sequence of QCTNYALLKLAGDVESNPGP (SEQ ID NO: 15); F2A has a sequence of VKQTLNFDLLKLAGDVESNPGP (SEQ ID NO: 16). Cleavage efficient 2A peptides suitable for rAAV vectors described herein are described in Chng J. et al. Mabs. 2015; 7(2):403-412, and Kim et al. PLoS One 2011; 6(4) the contents of each of which are hereby incorporated by reference in their entirety.

Adeno-associated virus (AAV): As used herein, the terms “adeno-associated virus” or “AAV” or recombinant AAV (“rAAV”) includes, but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV (see, e.g., Fields et al., Virology, volume 2, chapter 69 (4th ed., Lippincott-Raven Publishers); Gao et al., J. Virology 78:6381-6388 (2004); Mori et al., Virology 330:375-383 (2004)). Typically, AAV can infect both dividing and non-dividing cells and can be present in an extrachromosomal state without integrating into the genome of a host cell. AAV vectors are commonly used in gene therapy. In some embodiments, AAV are engineered. The AAV can be engineered through any methods known in the art. For example, in some embodiments, AAV capsids are engineered through protein engineering methods.

Administering: As used herein, the terms “administering,” or “introducing” are used interchangeably in the context of delivering rAAV vectors encoding an antibody into a subject, by a method or route which results in efficient delivery of the rAAV vector. Various methods are known in the art for administering rAAV vectors, including for example intravenously, subcutaneously or transdermally. Transdermal administration of rAAV vector can be performed by use of a “gene gun” or biolistic particle delivery system. In some embodiments, the rAAV vectors are administered via non-viral lipid nanoparticles.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antibody: As used herein, the term “antibody” or “Ab” or “Abs” or “mAbs” refers to immunoglobulin molecules and immunologically active portions of immunoglobulin (Ig) molecules, i.e., molecules that contain an antigen binding site that specifically binds (immunoreacts with) an antigen. By “specifically binds” or “immunoreacts with” it is meant that the antibody reacts with one or more regions of a desired antigen of the desired antigen. Antibodies include antibody fragments. Antibodies also include, but are not limited to, polyclonal, monoclonal, chimeric dAb (domain antibody), single chain, Fab, Fab′, F(ab′)2 fragments, scFvs, and Fₐb expression libraries. An antibody may be a whole antibody, or immunoglobulin, or an antibody fragment.

The recognized immunoglobulin polypeptides include the kappa and lambda light chains and the alpha, gamma (IgG1, IgG2, IgG3, IgG4), delta, epsilon and mu heavy chains or equivalents in other species. Full-length immunoglobulin “light chains” (of about 25 kDa or about 214 amino acids) comprise a variable region of about 110 amino acids at the NH2-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

Antigen binding site: As used herein, the term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

C1-esterase deficiency or C1-esterase disorder: As used herein, “C1-esterase deficiency” or “C1-esterase disorder” means a reduced amount of functional C1-esterase inhibitor present in a subject in comparison to a healthy individual.

Cleavable linker: As used herein, the term “cleavable linker” includes any polypeptide linker that is capable of being cleaved by a compound. For example, a cleavable linker can be a polypeptide linker that is enzymatically cleavable. Various enzymatically cleavable linkers are suitable for the present invention including for example furin-cleavable linkers or thrombin cleavable linkers.

Coupled, linked, joined, or fused: As used herein, the terms “coupled”, “linked”, “joined”, “fused”, and “fusion” are used interchangeably. These terms refer to the joining together of two more elements or components by whatever means, including chemical conjugation or recombinant means.

Epitope: As used herein, the term “epitope” includes any protein determinant capable of specific binding to an immunoglobulin, or fragment. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide.

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Hereditary angioedema or HAE: As used herein, the term “hereditary angioedema” or “HAE” refers to a blood disorder characterized by unpredictable and recurrent attacks of inflammation. HAE is typically associated with C1-INH deficiency, which may be the result of low levels of C1-INH or C1-INH with impaired or decreased activity. HAE is also associated with other genetic mutations, such as mutations in FXII among others. Symptoms include, but are not limited to, swelling that can occur in any part of the body, such as the face, extremities, genitals, gastrointestinal tract, and upper airways.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

IRES: As used herein, the term “IRES” refers to any suitable internal ribosome entry site sequence.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Immunological Binding: The term “immunological binding” refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein smaller K_{d} represents a greater affinity Immunological binding properties of selected polypeptides can be quantified using methods well known in the art.

Linker or peptide linker: The term “linker” or “peptide linker” as used herein refers to an amino acid sequence that connects two polypeptide domains. For example, a “linker” or “peptide linker” can separate an antibody heavy chain amino acid sequence and an antibody light chain amino acid sequence. Various kinds of linkers are suitable for the present invention, including for example, linkers that have a Gly-Ser-Gly (GSG) motif.

Polypeptide: The term, “polypeptide,” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Prevent: As used herein, the term “prevent” or “prevention”, when used in connection with the occurrence of a disease, disorder, and/or condition, refers to reducing the risk of developing the disease, disorder and/or condition.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding residues are homologous over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Suffering from: An individual who is “suffering from” a disease, disorder, and/or condition has been diagnosed with or displays one or more symptoms of the disease, disorder, and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

The recitation of numerical ranges by endpoints herein includes all numbers and fractions subsumed within that range (e.g. 1 to 5 includes 1, 1.5, 2, 2.75, 3, 3.9, 4 and 5). It is also to be understood that all numbers and fractions thereof are presumed to be modified by the term “about.”

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

Various aspects of the invention are described in detail in the following sections. The use of sections is not meant to limit the invention. Each section can apply to any aspect of the invention. In this application, the use of “or” means “and/or” unless stated otherwise.

### DETAILED DESCRIPTION

The present disclosure describes efficient and robust recombinant adeno-associated viral (rAAV) vectors that encode anti-plasma kallikrein antibodies for the treatment of plasma kallikrein-mediated disorders, such as HAE associated C1 INH deficiency. Human C1-INH is an important anti-inflammatory plasma protein with a wide range of inhibitory and non-inhibitory biological activities. By sequence homology, structure of its C-terminal domain, and mechanism of protease inhibition, it belongs to the serpin superfamily, the largest class of plasma protease inhibitors, which also includes antithrombin, α-proteinase inhibitor, plasminogen activator inhibitor, and many other structurally similar proteins that regulate diverse physiological systems. C1-INH is an inhibitor of proteases in the complement system, the contact system of kinin generation, and the intrinsic coagulation pathway.

Low plasma content of C1-INH or its dysfunction results in the activation of both complement and contact plasma cascades, and may affect other systems as well. A decrease in C1-INH plasma content to levels lower than 55 μg/mL (˜25% of normal) has been shown to induce spontaneous activation of C1. There are other manners whereby the kallikrein kinin system can become over activated even in the presence of normal C1-INH activity. For example, there are known mutations in Factor XII (FXII) that cause it to be more readily activated and thus more prone to subsequently activate prekallikrein into plasma kallikrein. In some embodiments, the rAAV vectors described herein are used to treat subjects with a disease or dysfunction mediated by excessive plasma kallikrein activity.

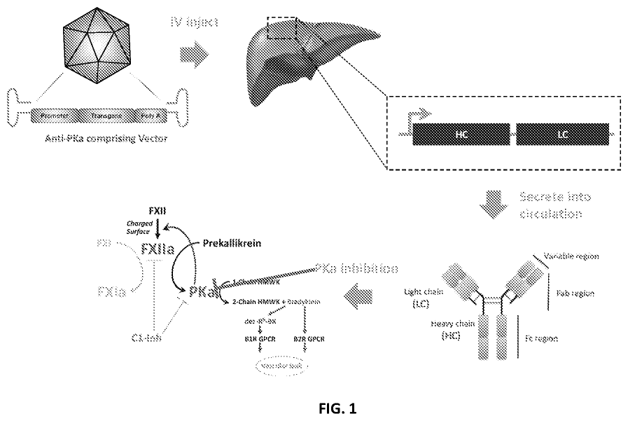

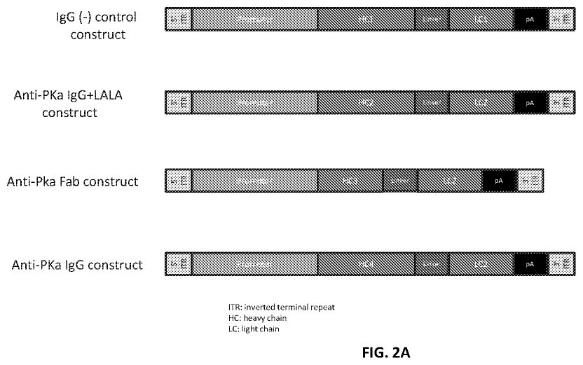

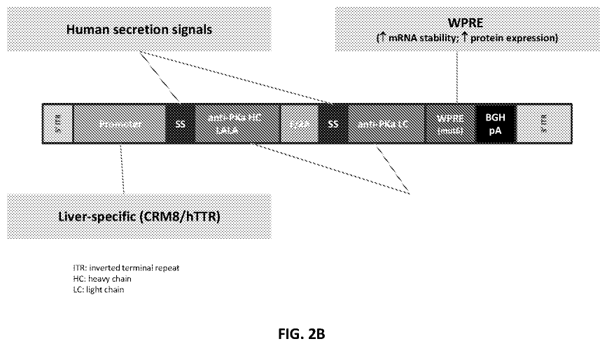

A schematic that illustrates the rAAV vector approach for the delivery of antibodies that bind to plasma kallikrein is depicted in FIG. 1. As shown in FIG. 1, an rAAV vector comprising a recombinant anti-plasma kallikrein antibody sequence is administered to a subject and results in the production of a fused heavy chain and light chain transcript. This transcript is subsequently cleaved resulting in the production of functional anti-plasma kallikrein antibodies that are secreted into the circulation. FIGS. 2A and 2B depict embodiments of an rAAV vector described herein.

Accordingly, the present disclosure provides, among other things, rAAV vectors that encode antibodies that are useful for the treatment of disease, such as diseases associated with kallikrein-kinin system disfunction. The rAAV vectors can be constructed to encode antibodies that target selected protein members of the kallikrein-kinin system, such as for example, plasma kallikrein.

In some embodiments, the rAAV vectors encode an anti-plasma kallikrein antibody. In some embodiments, the rAAV vector encodes an anti-plasma kallikrein antibody heavy chain and an anti-plasma kallikrein antibody light chain.

The present disclosure further provides, among other things, a method of treating a disease using the rAAV vectors described herein. In some embodiments, the disease is a disease associated with excessive activity of the kallikrein-kinin cascade, such as a C1-INH deficiency or disorder.

In some embodiments, the C1-INH deficiency or disorder is HAE.

rAAV Vector Design

In some aspects, provided herewith is a recombinant adeno-associated viral (rAAV) vector encoding an anti-plasma kallikrein antibody heavy chain and an anti-plasma kallikrein antibody light chain.

In some embodiments, the rAAV vector described herein produces a fused anti-plasma kallikrein antibody heavy chain and an anti-plasma kallikrein antibody light chain. The fused heavy chain and light chain transcript is subsequently cleaved to produce functional anti-plasma kallikrein antibodies that are secreted into the circulation. Accordingly, in some embodiments, the rAAV vector described herein provides one genetic cassette comprising both an anti-plasma kallikrein antibody heavy chain and an anti-plasma kallikrein antibody light chain sequence. In some embodiments, the liver acts as a depot following administration of the rAAV vector.

In some embodiments, a linker links the anti-plasma kallikrein antibody heavy chain and the anti-plasma kallikrein antibody light chain. Various kinds of linkers can be used in the rAAV vector. In some embodiments, the linker is a glycine/serine linker, i.e., a peptide linker consisting essentially of glycine and serine. In an exemplary embodiment, the linker comprises GS or GSG. In some embodiments, the linker is GSG. In another embodiment, the linker comprises the Gly-Ser-Gly (GSG) motif, such as GGSG (SEQ ID NO: 7), (GS)×3 (SEQ ID NO: 12), (GGSG)×2 (SEQ ID NO: 8), SGGSGGSGG (SEQ ID NO: 9), GGSGGGSGGGSG (SEQ ID NO: 10), (GGGGS)×3 (SEQ ID NO: 11).

In some embodiments, the linker is a cleavable linker. Numerous kinds of cleavable linkers are known in the art, for example those that are cleavable by enzymes. In some embodiments, the linker is a furin or thrombin cleavable linker. In some embodiments, the linker is a furin cleavable linker.

In some embodiments, the furin cleavable linker is followed by a 2A sequence. Various kinds of 2A sequences are known in the art, and include for example T2A, P2A, E2A or an F2A. In some embodiments, the 2A sequence is T2A. In some embodiments, the 2A sequence is P2A. In some embodiments, the 2A is E2A. In some embodiments, the 2A is F2A.

In some embodiments, the AAV vector has an IRES sequence. In some embodiments, the linker comprises an IRES sequence.

In some embodiments, the anti-plasma kallikrein antibody heavy chain and the anti-plasma kallikrein antibody light chain are controlled by a single promoter. Such a configuration would lead to the production of one fused heavy chain and light chain comprising transcript and, following cleavage of the fused heavy chain and light chain sequences, results in two polypeptide products.

In some embodiments, the anti-plasma kallikrein antibody heavy chain and the anti-plasma kallikrein antibody light chain are controlled by separate promoters.

Various kinds of promoters can be used in the rAAV vector described herein. These include, for example, ubiquitous, tissue-specific, and regulatable (e.g. inducible or repressible) promoters. In some embodiments, the promoter is modified. Various kinds of modified promoters are known in the art, and include for example, shortened minimal promoters among others. In some embodiments, the promotor is a ubiquitous promoter. In some embodiments, the promoter is a chicken beta actin promoter. In some embodiments, the promoter is a liver-specific promoter. Examples of suitable liver-specific promoters include human transthyretin promoter (TTR), modified hTTR (hTTR mod.), α-Antitrypsin promoter, Liver Promoter 1 (LP1), TRM promoter, human factor IX pro/liver transcription factor-responsive oligomers, LSP, CMV/CBA promoter (1.1 kb), CAG promoter (1.7 kb), mTTR, modified mTTR, mTTR pro, mTTR enhancer, and the basic albumin promoter. Liver specific promoters are described, for example, in Zhijian Wu et al., Molecular Therapy vol. 16, no 2, February 2008, the contents of which are incorporated herein by reference.

The rAAV vector can contain additional enhancer or regulatory elements to promote transcription and/or translation of the mRNA (e.g., enhancer sequences, Kozak sequences, polyadenylation sequences, transcriptional termination sequences, IRES and the like). In some embodiments, the vector comprises a 5′ and a 3′ inverted terminal repeat (ITR). In some embodiments, the vector comprises a one or more enhancer elements. In some embodiments, the vector comprises a poly(A) tail. In some embodiments, the rAAV vector comprises hepatic specific control elements/Regions (HCRs). In some embodiments, the rAAV vector comprises an ApoE Enhancer. In some embodiments, the rAAV vector comprises a Liver-specific nucleic acid regulatory element, such as for example a cis-regulatory element (CRE). CRE are described in EP 18202888, the contents of which are hereby incorporated by reference in its entirety. Exemplary CREs include for example CRE4 and CRE6. In some embodiments, CRE4 is used in combination with apolipoprotein A-II gene. In some embodiments, CRE6 is used in combination with apolipoprotein C-I gene.

In some embodiments, the rAAV vector comprises woodchuck hepatitis virus post-transcriptional control element (WPRE). Various optimized or variant forms of WPRE can be used with the vectors described herein, and include, for example WPRE wild-type, WPRE3, and WPREmut6delATG among others. WPRE and associated WPRE variants are described in U.S. Pat. Nos. 10,179,918; 7,419,829; 9,731,033; 8,748,169; 7,816,131; 8,865,881; 6,287,814; U.S. Patent Publication No. 2016/0199412; U.S. Patent Publication No. 2017/0114363; U.S. Patent Publication No. 2017/0360961; U.S. Patent Publication No. 2019/0032078; U.S. Patent Publication No. 2018/0353621; International Publication No. WO2017201527; International Publication No. WO2018152451; International Publication No. WO2013153361; International Publication No. WO2014144756; European Patent No. EP1017785; and European Patent Publication No. 3440191. Each of the foregoing publications are incorporated herein by reference in its entirety.

In some embodiments, the rAAV vector comprises a WPRE element, and/or clusters of transcription factor binding sites. Thus, in some embodiments, the rAAV vector comprises woodchuck hepatitis virus post-transcriptional control element (WPRE). In some embodiments, the rAAV vector comprises clusters of transcription factor binding sites.

In some embodiments, the rAAV vector comprises a cis regulatory module (CRM). Various kinds of CRM are suitable for use in the vectors described herein and include for example liver-specific CRM, neuronal-specific CRM and/or CRM8. Accordingly, in some embodiments, the CRM is a liver specific CRM. In some embodiments, the CRM is a neuronal-specific CFM. In some embodiments, the CRM is CRM8. In some embodiments, the vector includes more than one CRM. For example, in some embodiments, the vector comprises two, three, four, five or six CRMs. In some embodiments, the vector comprises three CRMs, for example three CRM8.

The rAAV vector comprises a secretion signal that is a naturally occurring and/or artificial signal peptide (e.g. recombinantly engineered). In some embodiments, the secretion signal is a naturally occurring signal peptide. In some embodiments, the secretion signal is an artificial signal peptide (e.g. recombinantly engineered). In some embodiments, the secretion signals are human secretion signals. In some embodiments, the secretion signals are murine secretion signals.

In some embodiments, the rAAV vector is sequence optimized to increase transcript stability, for more efficient translation, and to reduce immunogenicity. In some embodiments, the rAAV vector including the anti-plasma kallikrein heavy chain and light chains are sequence optimized to increase transcript stability, for more efficient translation, and to reduce immunogenicity. In some embodiments, the anti-plasma kallikrein heavy chain and light chains are codon optimized.

In some embodiments, the rAAV vector is an AAV1, AAV2, AAV3, AAV4, AAV5, AAV6, AAV7, AAV8, AAV9, AAV10, or AAV11 vector. In some embodiments, the rAAV vector is AAV 1. In some embodiments, the rAAV vector is AAV 2. In some embodiments, the rAAV vector is AAV 3. In some embodiments, the rAAV vector is AAV 4. In some embodiments, the rAAV vector is AAV 5. In some embodiments, the rAAV vector is AAV 6. In some embodiments, the rAAV vector is AAV 7. In some embodiments, the rAAV vector is AAV 8. In some embodiments, the rAAV vector is AAV 9. In some embodiments, the rAAV vector is AAV 10. In some embodiments, the rAAV vector is AAV 11.

In some aspects, provided herewith is a nucleic acid comprising a nucleotide sequence encoding an anti-plasma kallikrein antibody heavy chain and an anti-plasma kallikrein antibody light chain. In some embodiments, the nucleic acid is DNA. In some embodiments, the nucleic acid is RNA. In some embodiments, the nucleic acid is combination of DNA and RNA. In some embodiments, provided herewith is a vector comprising a nucleotide sequence encoding an anti-plasma kallikrein antibody heavy chain and an anti-plasma kallikrein antibody light chain.

In some embodiments, the nucleotide sequence is operatively linked to a promoter. In some embodiments, the promoter is a liver-specific promoter. Examples of liver-specific promoters include human transthyretin promoter (TTR) and modified hTTR, (hTTR mod.). Various suitable promoters that can be used in various embodiments are described above.

In some embodiments, the nucleotide sequence is operatively linked to a cis-actin regulatory module (CRM). In some embodiments, the CRM includes a liver-specific CRM. Some embodiments include three CRM, for example three CRM8. Various kinds of suitable CRMs that can be used in various embodiments are described herein.

In some embodiments, the nucleotide sequence is operatively linked to a woodchuck hepatitis virus post-transcriptional control element (WPRE). In some embodiments, the WPRE is a WPREmut6. Various optimized or variant forms of WPRE are known in the art, and have been described herein.

In some embodiments, the nucleotide sequence is operatively linked to a secretion signal that is a naturally occurring or an artificial signal peptide (e.g. recombinantly engineered). In some embodiments, the secretion signal is a naturally occurring signal peptide. In some embodiments, the secretion signal is an artificial signal peptide (e.g. recombinantly engineered). In some embodiments, the secretion signals are human secretion signals. In some embodiments, the secretion signals are murine secretion signals.

### Anti-Plasma Kallikrein Antibodies

Exemplary heavy chain and light chain anti-plasma kallikrein amino acid sequences encoded by the rAAV vector are shown in the Table 1 below.

In some embodiments, the anti-plasma kallikrein antibodies are engineered to have extended half-life. To this end, in some embodiments, the anti-plasma kallikrein antibodies comprise an NHance mutation (i.e., H433K and N434F). In some embodiments, the anti-plasma kallikrein antibodies comprise YTE mutations (i.e., M252Y/S254T/T256E).

In some embodiments, the anti-plasma kallikrein antibodies are engineered to have reduced interactions with Fc receptors. To this end, in some embodiments, the anti-plasma kallikrein antibodies comprise a LALA mutation (i.e., L234A and L235A).

In some embodiments, the anti-plasma kallikrein antibodies are fused to albumin or an FcRn interacting peptide.

In some embodiments, the heavy chain and the light chain sequences are about 50%, 55%, 60%, 65%, 70%, 75%, 80%, 85%, 90%, 95%, or 100% identical to the sequences described in Table below. In some embodiments, the heavy chain and the light chain sequences are about 50% identical to the sequences described in Table 1. In some embodiments, the heavy chain and the light chain sequences are about 55% identical to the sequences described in Table 1. In some embodiments, the heavy chain and the light chain sequences are about 60% identical to the sequences described in Table 1. In some embodiments, the heavy chain and the light chain sequences are about 65% identical to the sequences described in Table 1. In some embodiments, the heavy chain and the light chain sequences are about 70% identical to the sequences described in Table 1. In some embodiments, the heavy chain and the light chain sequences are about 75% identical to the sequences described in Table 1. In some embodiments, the heavy chain and the light chain sequences are about 80% identical to the sequences described in Table 1. In some embodiments, the heavy chain and the light chain sequences are about 85% identical to the sequences described in Table 1. In some embodiments, the heavy chain and the light chain sequences are about 90% identical to the sequences described in Table 1. In some embodiments, the heavy chain and the light chain sequences are about 95% identical to the sequences described in Table 1. In some embodiments, the heavy chain and the light chain sequences are about 100% identical to the sequences described in Table 1. In some embodiments, the heavy chain and the light chain sequences identical to the sequences described in the Table 1.

TABLE 1
Exemplary anti-plasma kallikrein heavy chain and light chain
amino acid sequences

Anti-plasma kallikrein mature heavy chain sequence (no
secretion signal)
EVQLLESGGGLVQPGGSLRLSCAASGFTFSHYIMMWVRQAPGKGLEWVSGIYSSGGITVYA
DSVKGRFTISRDNSKNTLYLQMNSLRAEDTAVYYCAYRRIGVPRRDEFDIWGQGTMVTVSS
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPELLGGPS
VFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQGNV
FSCSVMHEALHNHYTQKSLSLSPG
(SEQ ID NO: 1)
Anti-plasma kallikrein mature light chain sequence (no
secretion signal)
DIQMTQSPSTLSASVGDRVTITCRASQSISSWLAWYQQKPGKAPKLLIYKASTLESGVPSR
FSGSGSGTEFTLTISSLQPDDFATYYCQQYNTYWTFGQGTKVEIKRTVAAPSVFIFPPSDE
QLKSGTASVVCLLNNFYPREAKVQWKVDNALQSGNSQESVTEQDSKDSTYSLSSTLTLSKA
DYEKHKVYACEVTHQGLSSPVTKSFNRGEC
(SEQ ID NO: 2)
Anti-plasma kallikrein mature heavy chain sequence (no
secretion signal) with LALA mutation
EVQLLESGGGLVQPGGSLRLSCAASGFTFSHYIMMWVRQAPGKGLEWVSGIYSSGGITVYA
DSVKGRFTISRDNSKNTLYLQMNSLRAEDTAVYYCAYRRIGVPRRDEFDIWGQGTMVTVSS
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPEAAGGPS
VFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQGNV
FSCSVMHEALHNHYTQKSLSLSPG
(SEQ ID NO: 3)
* Underline indicates LALA mutation amino acids

In some embodiments, exemplary anti-plasma kallikrein antibodies have a heavy chain CDR1 comprising FTFSHYIMM (SEQ ID NO: 17). In some embodiments, exemplary anti-plasma kallikrein antibodies have a heavy chain CDR2 comprising GIYSSGGITVYADSVKGRFTI (SEQ ID NO: 18). In some embodiments, exemplary anti-plasma kallikrein antibodies have a heavy chain CDR3 comprising RRIGVPRRDEFDI (SEQ ID NO: 19). In some embodiments, exemplary anti-plasma kallikrein antibodies have a heavy chain CDR1 comprising FTFSHYIMM (SEQ ID NO: 17), a CDR2 comprising GIYSSGGITVYADSVKGRFTI (SEQ ID NO: 18), and a CDR3 comprising RRIGVPRRDEFDI (SEQ ID NO: 19).

In some embodiments, exemplary anti-plasma kallikrein antibodies have a light chain CDR1 comprising RASQSISSWLA (SEQ ID NO: 20). In some embodiments, exemplary anti-plasma kallikrein antibodies have a light chain CDR2 comprising YKASTLESGVPSRF (SEQ ID NO: 21). In some embodiments, exemplary anti-plasma kallikrein antibodies have a light chain CDR3 comprising QQYNTYWT (SEQ ID NO: 22). In some embodiments, exemplary anti-plasma kallikrein antibodies have a light chain CDR1 comprising RASQSISSWLA (SEQ ID NO: 20), a light chain CDR2 comprising (SEQ ID NO: 21), and a light chain CDR3 comprising QQYNTYWT (SEQ ID NO: 22).

In some embodiments, exemplary anti-plasma kallikrein antibodies have a heavy chain CDR1 comprising FTFSHYIMM (SEQ ID NO: 17), a CDR2 comprising GIYSSGGITVYADSVKGRFTI (SEQ ID NO: 18), and a CDR3 comprising RRIGVPRRDEFDI (SEQ ID NO: 19). In some embodiments, exemplary anti-plasma kallikrein antibodies have a light chain CDR1 comprising RASQSISSWLA (SEQ ID NO: 20), a light chain CDR2 comprising YKASTLESGVPSRF (SEQ ID NO: 21), and a light chain CDR3 comprising QQYNTYWT (SEQ ID NO: 22).

In some embodiments, the CDRs disclosed herein have 1, 2, 3, or 4 amino acid substitutions, deletions or insertions in relation to the CDRs recited herein. In some embodiments, the CDRs disclosed herein contain no more than 3, 2 or 1 amino acid substitutions, deletions or insertions in comparison to the recited CDR sequence. In some embodiments, affinity maturated variants are obtained with desirable binding properties. Various affinity matured CDR sequences are presented in WO2014152232, the contents of which are hereby incorporated by reference in its entirety.

Exemplary anti-plasma kallikrein antibodies of the present disclosure include, without limitation, IgG (e.g., IgG1, IgG2, IgG3, and IgG4), IgM, IgA (e.g., IgA1, IgA2, and IgAsec), IgD, IgE, Fab, Fab′, Fab′2, F(ab′)2, Fd, Fv, Feb, scFv, scFv-Fc, and SMIP binding moieties. In certain embodiments, the anti-plasma kallikrein antibody encodes the heavy chain and the light chain sequences of Lanadelumab. In some embodiments, the antibody is a full length antibody. In some embodiments, the antibody is not an antibody fragment. In some embodiments, the antibody is not an Fab.

In certain embodiments, the antibody is an scFv. The scFv may include, for example, a flexible linker allowing the scFv to orient in different directions to enable antigen binding. In various embodiments, the antibody may be a cytosol-stable scFv or intrabody that retains its structure and function in the reducing environment inside a cell (see, e.g., Fisher and DeLisa, J. Mol. Biol. 385(1): 299-311, 2009; incorporated by reference herein). In particular embodiments, the scFv is converted to an IgG or a chimeric antigen receptor according to the methods described herein. In embodiments, the antibody binds to both denatured and native protein targets. In embodiments, the antibody binds to either denatured or native protein. In some embodiments, the antibody binds a select member of the complement system. In some embodiments, the antibody binds to plasma kallikrein.

In most mammals, including humans, whole antibodies have at least two heavy (H) chains and two light (L) chains connected by disulfide bonds. Each heavy chain consists of a heavy chain variable region (VH) and a heavy chain constant region (CH). The heavy chain constant region consists of three domains (CH1, CH2, and CH3) and a hinge region between CH1 and CH2. Each light chain consists of a light chain variable region (VL) and a light chain constant region (CL). The light chain constant region consists of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

Antibodies include all known forms of antibodies and other protein scaffolds with antibody-like properties. For example, the antibody can be a monoclonal antibody, a polyclonal antibody, human antibody, a humanized antibody, a bispecific antibody, a monovalent antibody, a chimeric antibody, or a protein scaffold with antibody-like properties, such as fibronectin or ankyrin repeats. The antibody can have any of the following isotypes: IgG (e.g., IgG1, IgG2, IgG3, and IgG4), IgM, IgA (e.g., IgA1, IgA2, and IgAsec), IgD, or IgE.

An antibody fragment may include one or more segments derived from an antibody. A segment derived from an antibody may retain the ability to specifically bind to a particular antigen. An antibody fragment may be, e.g., a Fab, Fab′, Fab′2, F(ab′)2, Fd, Fv, Feb, scFv, or SMIP. An antibody fragment may be, e.g., a diabody, triabody, affibody, nanobody, aptamer, domain antibody, linear antibody, single-chain antibody, or any of a variety of multispecific antibodies that may be formed from antibody fragments.

Examples of antibody fragments include: (i) a Fab fragment: a monovalent fragment consisting of VL, VH, CL, and CH1 domains; (ii) a F(ab′)2 fragment: a bivalent fragment comprising two Fab fragments linked by a disulfide bridge at the hinge region; (iii) a Fd fragment: a fragment consisting of VH and CH1 domains; (iv) an Fv fragment: a fragment consisting of the VL and VH domains of a single arm of an antibody; (v) a dAb fragment: a fragment including VH and VL domains; (vi) a dAb fragment: a fragment that is a VH domain; (vii) a dAb fragment: a fragment that is a VL domain; (viii) an isolated complementarity determining region (CDR); and (ix) a combination of two or more isolated CDRs which may optionally be joined by one or more synthetic linkers. Furthermore, although the two domains of the Fv fragment, VL and VH, are coded for by separate genes, they can be joined, using recombinant methods, e.g., by a synthetic linker that enables them to be expressed as a single protein, of which the VL and VH regions pair to form a monovalent binding moiety (known as a single chain Fv (scFv)). Antibody fragments may be obtained using conventional techniques known to those of skill in the art, and may, in some instances, be used in the same manner as intact antibodies. Antigen-binding fragments may be produced by recombinant DNA techniques or by enzymatic or chemical cleavage of intact immunoglobulins. An antibody fragment may further include any of the antibody fragments described above with the addition of additional C-terminal amino acids, N-terminal amino acids, or amino acids separating individual fragments.

An antibody may be referred to as chimeric if it includes one or more antigen-determining regions or constant regions derived from a first species and one or more antigen-determining regions or constant regions derived from a second species. Chimeric antibodies may be constructed, e.g., by genetic engineering. A chimeric antibody may include immunoglobulin gene segments belonging to different species (e.g., from a mouse and a human).

Use of rAAV Vectors that Encode Anti-Plasma Kallikrein Antibody for Treatment of Disease

Described herein are methods of treating a disease associated with unregulated plasma kallikrein activity, such as a deficiency or disorder in C1 esterase inhibitor, in a subject in need thereof comprising administering an AAV vector that encodes an anti-plasma kallikrein antibody heavy chain and an anti-plasma kallikrein antibody light chain. Following administration of the rAAV vector described herein, the anti-plasma kallikrein antibody heavy chain and the light chain assemble into a functional antibody. The functional antibody is secreted into the circulation and binds plasma kallikrein.

The rAAV vector described herein can be used to treat any C1 esterase inhibitor deficiency or disorder and/or disorder mediated by dysregulated plasma kallikrein activity. In some embodiments, the disorder is hereditary angioedema (HAE), acquired angioedema (AAE), rheumatoid arthritis, gout, intestinal bowel disease, oral mucositis, neuropathic pain, inflammatory pain, spinal stenosis-degenerative spine disease, arterial or venous thrombosis, post operative ileus, aortic aneurysm, osteoarthritis, vasculitis, edema, cerebral edema, pulmonary embolism, stroke, clotting induced by ventricular assistance devices or stents, head trauma or peri-tumor brain edema, sepsis, acute middle cerebral artery (MCA) ischemic event, restenosis, systemic lupus erythematosis nephritis/vasculitis, diabetic macular edema, or burn injury. In some embodiments, the C1 esterase inhibitor deficiency or disorder is HAE. The HAE can be any kind of HAE, including HAE type I, II, or III.

In some embodiments, the rAAV vector remains episomal following administration to a subject in need thereof. In some embodiments, the rAAV vector does not remain episomal following administration to a subject in need thereof. For example, in some embodiments, the rAAV vector integrates into the genome of the subject. Such integration can be achieved, for example, by using various gene-editing technologies, such as, zinc finger nucleases (ZFNs), Transcription activator-like effector nucleases (TALENS), ARCUS genome editing, and/or CRISPR-Cas systems.

In some embodiments, a pharmaceutical composition comprising an rAAV vector described herein is used to treat subjects in need thereof. The pharmaceutical composition containing an rAAV vector or particle of the invention contains a pharmaceutically acceptable excipient, diluent or carrier. Examples of suitable pharmaceutical carriers are well known in the art and include phosphate buffered saline solutions, water, emulsions, such as oil/water emulsions, various types of wetting agents, sterile solutions and the like. Such carriers can be formulated by conventional methods and are administered to the subject at a therapeutically effective amount.

The rAAV vector is administered to a subject in need thereof via a suitable route. In embodiments, the rAAV vector is administered by intravenous, intraperitoneal, subcutaneous, or intradermal administration. In embodiments, the rAAV vector is administered intravenously. In embodiments, the intradermal administration comprises administration by use of a “gene gun” or biolistic particle delivery system. In some embodiments, the rAAV vector is administered via a non-viral lipid nanoparticle. For example, a composition comprising the rAAV vector may comprise one or more diluents, buffers, liposomes, a lipid, a lipid complex. In some embodiments, the rAAV vector is comprised within a microsphere or a nanoparticle, such as a lipid nanoparticle.

In some embodiments, functional anti-plasma kallikrein antibody is detectable in plasma of the subject at about 2 to 6 weeks post administration of the rAAV vector. In some embodiments, functional anti-plasma kallikrein antibody is detectable in plasma of the subject at about 2 weeks. In some embodiments, functional anti-plasma kallikrein antibody is detectable in plasma of the subject at about 3 weeks. In some embodiments, functional anti-plasma kallikrein antibody is detectable in plasma of the subject at about 4 weeks. In some embodiments, functional anti-plasma kallikrein antibody is detectable in plasma of the subject at about 5 weeks. In some embodiments, functional anti-plasma kallikrein antibody is detectable in plasma of the subject at about 6 weeks. In some embodiments, functional anti-plasma kallikrein antibody is detectable in hepatocytes of the subject at about 2 to 6 weeks post administration of the rAAV vector.

In some embodiments, functional anti-plasma kallikrein antibody is detectable in plasma of the subject at least 3 months, 6 months, 12 months, 2 years, 3 years, 4 years, 5 years, 6 years, 7 years, 8 years, 9 years, or 10 years after administration of the rAAV vector. Accordingly, in some embodiments, functional anti-plasma kallikrein antibody is detectable in plasma of the subject at least 3 months after administration of the rAAV vector. In some embodiments, functional anti-plasma kallikrein antibody is detectable in plasma of the subject at least 6 months after administration of the rAAV vector. In some embodiments, functional anti-plasma kallikrein antibody is detectable in plasma of the subject at least 12 months after administration of the rAAV vector. In some embodiments, functional anti-plasma kallikrein antibody is detectable in plasma of the subject at least 2 years after administration of the rAAV vector. In some embodiments, functional anti-plasma kallikrein antibody is detectable in plasma of the subject at least 3 years after administration of the rAAV vector. In some embodiments, functional anti-plasma kallikrein antibody is detectable in plasma of the subject at least 4 years after administration of the rAAV vector. In some embodiments, functional anti-plasma kallikrein antibody is detectable in plasma of the subject at least 5 years after administration of the rAAV vector. In some embodiments, functional anti-plasma kallikrein antibody is detectable in plasma of the subject at least 6 years after administration of the rAAV vector. In some embodiments, functional anti-plasma kallikrein antibody is detectable in plasma of the subject at least 7 years after administration of the rAAV vector. In some embodiments, functional anti-plasma kallikrein antibody is detectable in plasma of the subject at least 8 years after administration of the rAAV vector. In some embodiments, functional anti-plasma kallikrein antibody is detectable in plasma of the subject at least 9 years after administration of the rAAV vector. In some embodiments, functional anti-plasma kallikrein antibody is detectable in plasma of the subject at least 10 years after administration of the rAAV vector. In some embodiments, functional anti-plasma kallikrein antibody is detectable in plasma of the subject for the remainder of the subject's life following administration of the rAAV vector. In some embodiments, the administered rAAV comprising anti-plasma kallikrein antibody heavy chain and anti-plasma kallikrein antibody light chain antibody results in the production of active anti-PKa antibody to the same extent as found following administration of purified anti-PKa IgG delivered intravenously. In some embodiments, the administered rAAV comprising anti-plasma kallikrein antibody heavy chain and anti-plasma kallikrein antibody light chain antibody results in production of a greater amount of active anti-PKa antibody as compared to administration of purified anti-PKa IgG delivered intravenously.

In some embodiments, the administered rAAV comprising anti-plasma kallikrein antibody heavy chain and anti-plasma kallikrein antibody light chain antibody results in the production of at least 60% active anti-PKa antibody. In some embodiments, the administered rAAV comprising anti-plasma kallikrein antibody heavy chain and anti-plasma kallikrein antibody light chain antibody results in the production of at least 65% active anti-PKa antibody. In some embodiments, the administered rAAV comprising anti-plasma kallikrein antibody heavy chain and anti-plasma kallikrein antibody light chain antibody results in the production of at least 70% active anti-PKa antibody. In some embodiments, the administered rAAV comprising anti-plasma kallikrein antibody heavy chain and anti-plasma kallikrein antibody light chain antibody results in the production of at least 75% active anti-PKa antibody. In some embodiments, the administered rAAV comprising anti-plasma kallikrein antibody heavy chain and anti-plasma kallikrein antibody light chain antibody results in the production of at least 80% active anti-PKa antibody. In some embodiments, the administered rAAV comprising anti-plasma kallikrein antibody heavy chain and anti-plasma kallikrein antibody light chain antibody results in the production of at least 85% active anti-PKa antibody. In some embodiments, the administered rAAV comprising anti-plasma kallikrein antibody heavy chain and anti-plasma kallikrein antibody light chain antibody results in the production of at least 90% active anti-PKa antibody. In some embodiments, the administered rAAV comprising anti-plasma kallikrein antibody heavy chain and anti-plasma kallikrein antibody light chain antibody results in the production of at least 95% active anti-PKa antibody. In some embodiments, the administered rAAV comprising anti-plasma kallikrein antibody heavy chain and anti-plasma kallikrein antibody light chain antibody results in the production of at least 99% active anti-PKa antibody.

In some embodiments, following administration of the AAV vector to the subject the levels of plasma kallikrein IgG detectable in the circulation are between about 4 and 10 times greater than IgG detectable following direct administration of purified plasma kallikrein antibody to the subject. In some embodiments, following administration of the AAV vector to the subject the levels of active plasma kallikrein IgG detectable meets or exceeds human therapeutic level. In some embodiments, the levels of active plasma kallikrein IgG post administration of the rAAV vector is about between 2 and 35 times the human therapeutic level. In some embodiments, the levels of active plasma kallikrein IgG post administration is about 2 times the human therapeutic level. In some embodiments, the levels of active plasma kallikrein IgG post administration is about 3 times the human therapeutic level. In some embodiments, the levels of active plasma kallikrein IgG post administration is about 4 times the human therapeutic level. In some embodiments, the levels of active plasma kallikrein IgG post administration is about 5 times the human therapeutic level. In some embodiments, the levels of active plasma kallikrein IgG post administration is about 6 times the human therapeutic level. In some embodiments, the levels of active plasma kallikrein IgG post administration is about 6 times the human therapeutic level. In some embodiments, the levels of active plasma kallikrein IgG post administration is about 7 times the human therapeutic level. In some embodiments, the levels of active plasma kallikrein IgG post administration is about 8 times the human therapeutic level. In some embodiments, the levels of active plasma kallikrein IgG post administration is about 9 times the human therapeutic level. In some embodiments, the levels of active plasma kallikrein IgG post administration is about 10 times the human therapeutic level. In some embodiments, the levels of active plasma kallikrein IgG post administration is about 15 times the human therapeutic level. In some embodiments, the levels of active plasma kallikrein IgG post administration is about 20 times the human therapeutic level. In some embodiments, the levels of active plasma kallikrein IgG post administration is about 25 times the human therapeutic level. In some embodiments, the levels of active plasma kallikrein IgG post administration is about 30 times the human therapeutic level. In some embodiments, the levels of active plasma kallikrein IgG post administration is about 35 times the human therapeutic level.

Thus, administration of rAAV vector comprising the anti-plasma kallikrein antibody heavy chain and an anti-plasma kallikrein antibody light chain results in sustained robust expression in comparison to a single administration of purified anti-plasma kallikrein antibody to a subject in need.

In some embodiments, the administered rAAV vector produces anti-plasma kallikrein antibodies that are capable of inhibiting plasma kallikrein activity by between about 50 and 95%. Thus, in some embodiments, the administered rAAV vector produces anti-plasma kallikrein antibodies that are capable of inhibiting plasma kallikrein activity by about 50%. In some embodiments, the administered rAAV vector produces anti-plasma kallikrein antibodies that are capable of inhibiting plasma kallikrein activity by about 55%. In some embodiments, the administered rAAV vector produces anti-plasma kallikrein antibodies that are capable of inhibiting plasma kallikrein activity by about 60%. In some embodiments, the administered rAAV vector produces anti-plasma kallikrein antibodies that are capable of inhibiting plasma kallikrein activity by about 65%. In some embodiments, the administered rAAV vector produces anti-plasma kallikrein antibodies that are capable of inhibiting plasma kallikrein activity by about 70%. In some embodiments, the administered rAAV vector produces anti-plasma kallikrein antibodies that are capable of inhibiting plasma kallikrein activity by about 75%. In some embodiments, the administered rAAV vector produces anti-plasma kallikrein antibodies that are capable of inhibiting plasma kallikrein activity by about 75%. In some embodiments, the administered rAAV vector produces anti-plasma kallikrein antibodies that are capable of inhibiting plasma kallikrein activity by about 80%. In some embodiments, the administered rAAV vector produces anti-plasma kallikrein antibodies that are capable of inhibiting plasma kallikrein activity by about 85%. In some embodiments, the administered rAAV vector produces anti-plasma kallikrein antibodies that are capable of inhibiting plasma kallikrein activity by about 90%. In some embodiments, the administered rAAV vector produces anti-plasma kallikrein antibodies that are capable of inhibiting plasma kallikrein activity by about 95%.

### EXAMPLES

Other features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

### Example I. Vector Design

Exemplary methods and designs of generating rAAV expression constructs (rAAV vectors) comprising coding sequences of anti-kallikrein antibody and variations of the same are provided in this Example. In this study, recombinant AAV vector (rAAV8) was used. The basic design of an rAAV vector comprises of an expression cassette flanked by inverted terminal repeats (ITRs): a 5′-ITR and a 3′-ITR. These ITRs mediate the replication and packaging of the vector genome by the AAV replication protein Rep and associated factors in vector producer cells. Typically an expression cassette contains a promoter, a coding sequence, a polyA tail and/or a tag. An expression construct encoding human anti-plasma kallikrein (PKa)-IgG antibody (lanadelumab) was designed and prepared using standard molecular biology techniques. The coding sequence for the anti-PKa antibody heavy chain (HC) and the coding sequence for the anti-PKa antibody light chain (LC) were inserted downstream of a promoter, the chicken B-actin promoter (CB). In another exemplary method and design, the promoter (+/−enhancer) is a liver-specific promoter comprisingCRM8/hTTR. In some embodiments, the expression cassette also includes a WPRE element and human secretion signals (SS). A short linker comprising oligonucleotides encoding a furin cleavable site (F/2A) was inserted between the HC and LC. A 168 bp SV40 pol A sequence and a DNA titer sequence were inserted downstream of the IgG LC. FIGS. 2A and 2B exemplify the schematic representations of the expression constructs. The expression constructs were then ligated to the AAV vector and tested by sequencing. Vectors were packaged in viral particles and stored.

Any number of variations of the above scheme can be performed. Alternative constructs can be obtained by replacing the coding sequences for HC and LC with coding sequence for fragment antigen binding (Fab); replacing the anti-PKa coding sequence with variant having the leucine-to-alanine mutation (LALA) that prevent the interaction with Fc receptors (FIGS. 2A and 2B). Additionally, more than one promoter may be used, and/or an IRES sequence may be introduced upstream of the LC.

### Example 2. Expression of rAAV-Driven Anti-PKa Antibody In Vivo

Exemplary studies described below are directed to test the rAAV-driven expression of the anti-PKA antibody. Mice were injected with rAAV vectors expressing (a) negative control vector ((−) ye control); or test samples (b) anti-PKa IgG+LALA construct, (c) anti-PKa Fab; (d) anti-PKa IgG; or a positive control (purified anti-plasma kallikrein antibody) as described in Table 2.

TABLE 2
Exemplary in vivo study using rAAV vectors
that encode anti-plasma kallikrein antibody

| Group | Condition | Treatment | Volume | Dose | N/group |
|---|---|---|---|---|---|
| 1 | Negative | rAAV8-null ctrl | 200 μl | 1E11gc | 5 |
| | control | | | | |
| 2 | Test | rAAV8- ctrl IgG | 200 μl | 1E11gc | 5 |
| 3 | Test | rAAV8- PKa-LALA IgG | 200 μl | 1E11gc | 5 |
| 4 | Test | rAAV8- PKa Fab | 200 μl | 1E11gc | 5 |
| 5 | Test | rAAV8- PKa IgG | 200 μl | 1E11gc | 5 |
| 6 | Positive | PKa LALA IgG; IV ctrl | ~50 μl | 10 mg/kg | 4 |
| | control | | | protein | |
| 7 | Negative | Uninjected | — | — | 3 |
| | control | | | | |

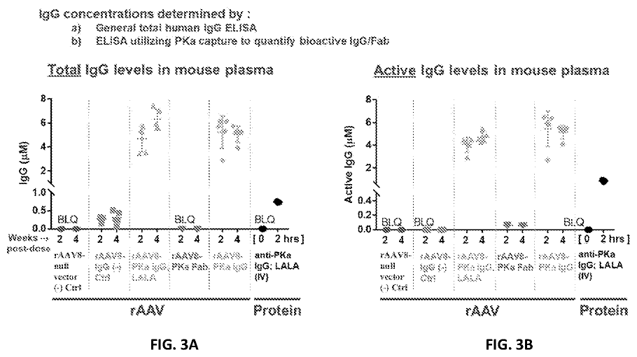

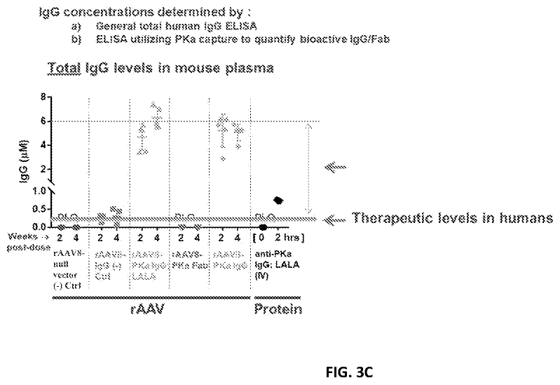

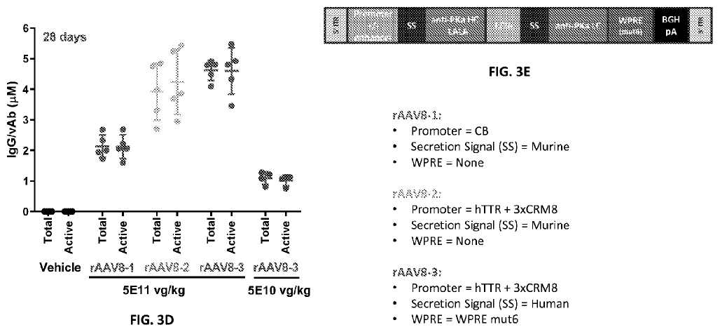

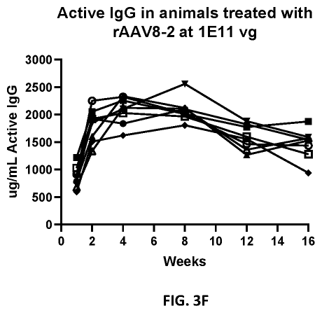

Plasma was collected at 2 weeks and 4 weeks after injection of rAAV, and total human IgG concentration in plasma was determined by ELISA. Results are depicted in FIG. 3A-3C. Mice injected with control rAAV did not show any increase in total human IgG levels. On the other hand total human IgG concentration in plasma was high in mice receiving anti-PKa IgG+LALA and anti-PKa IgG (FIG. 3A-3C). Similar results were obtained when the level of active IgG was tested as shown in FIGS. 3A and 3B. Further, FIG. 3C demonstrated that the level of increase of the anti-PKa IgG antibodies after injection with rAAV constructs was nearly 30 times greater than the therapeutic levels in humans.

Another exemplary study described below assessed anti-PKa antibody expression by rAAV expression constructs comprising one or more of CRM8/hTTR, secretion signal, and WPRE elements. Three rAAV expression constructs—rAAV8-1, rAAV8-2, and rAAV8-3—were administered via tail vein injection to mice at a 5×10¹¹ vg/kg dose. The level of expression of rAAV8-3 construct was tested at two vector doses—5×10¹¹ vg/kg and 5×10¹⁰ vg/kg. Vector rAAV8-1 comprises the chicken B-actin promoter (CB) and murine secretion signals, but no WPRE element. Vector rAAV8-2 comprises hTTR+3xCRM8 promoter and murine secretion signals, but no WPRE element. Vector rAAV8-3 comprises hTTR+3xCRM8 promoter, and human secretion signals, and WPREmut6 element.

Plasma was collected at 28 days after injection of rAAV, and total human IgG and active human IgG levels in plasma were determined by ELISA. Results are depicted in FIG. 3D. Mice injected with vehicle only did not show any increase in human IgG levels. On the other hand, total and active human IgG concentrations in plasma were high in mice receiving rAAV8-1, rAAV8-2, and rAAV8-3 constructs. Both total and active human IgG levels in plasma were the higher in mice that received rAAV8-2 and rAAV8-3 constructs compared to that of rAAV8-1, indicating the contribution of liver-specific promoter and enhancer elements in boosting the IgG expression. Both total and active human IgG levels in plasma of mice receiving rAAV8-3 construct were not only higher compared to that of mice receiving rAAV8-2 construct but they were also relatively stable indicating the contribution of WPRE element in stabilizing the mRNA. Stable expression of IgG levels in plasma of mice receiving rAAV8-3 construct is also evident when the rAAV8-3 constructs were injected into mice at a lower dose, 5×10¹⁰ vg/kg. The IgG expression data shown in FIG. 3D for rAAV8-3 construct indicates a stable IgG expression.

Another exemplary study described below is directed to test the duration of anti-PKA antibody expression by rAAV8-2 construct in mice. rAAV8-2 construct was to mice via tail vein injection (n=8) at a 1×10¹¹ vg/kg dose, and level of active IgG expression in plasma was measured over a period of 16 weeks (4 months). As described above, vector rAAV8-2 comprises hTTR+3xCRM8 promoter and murine secretion signals, but no WPRE element.

Plasma was collected at 2, 4, 6, 8, 10, 12, 14, and 16 weeks after injection of rAAV8-2, and active human IgG levels in plasma was determined by ELISA. Mice injected with rAAV8-2 vector continued to express active human IgG for over 16 weeks. Expression profiles of active human IgG in mice plasma for up to 16 weeks are depicted in FIG. 3F.

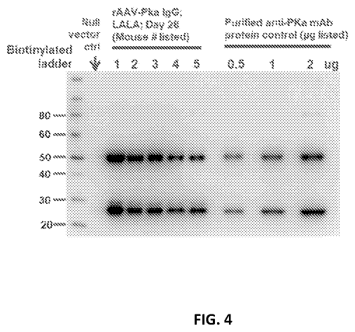

FIG. 4 shows successful processing and expression of anti-PKa IgG+LALA heavy and light chain proteins in murine plasma samples collected at Day 28. Western blot was performed after electrophoresing the samples in a reducing 8-6% Tris-glycine gel, and immunoblot reaction was performed using rabbit anti-human IgG H & L antibody at a dilution of 1:5000.

### Example 3. Inhibition of PKa by rAAV-Driven Anti-PKa Antibody

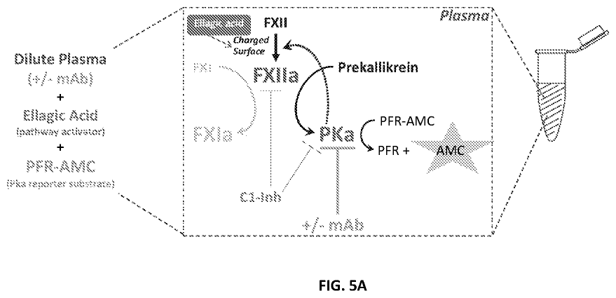

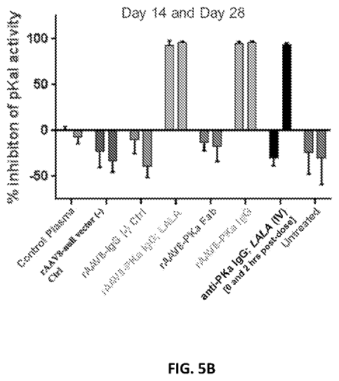

In order to determine the level of PKa inhibition a fluorogenic assay was used, as depicted in the schematic of FIG. 5A. Plasma was collected from mice injected with control or anti-PKa antibody-expressing rAAV at 14 days and 28 days after injection. As demonstrated in FIG. 5B, mice injected with anti-PKa IgG+LALA and anti-PKa IgG showed robust inhibition of PKa activity at Day 14 and Day 28.

### Example 4: Expression of rAAV8/Anti-PKa IgG or Fab in Mouse Livers

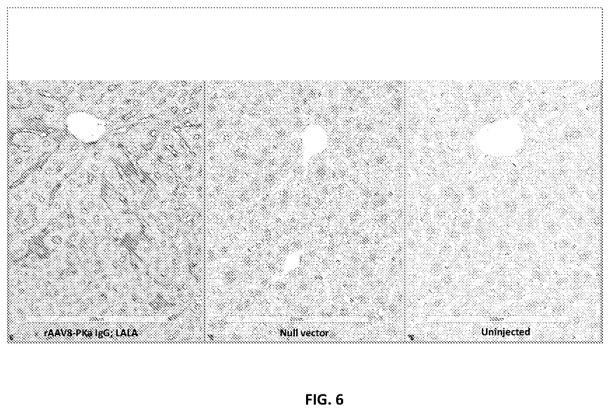

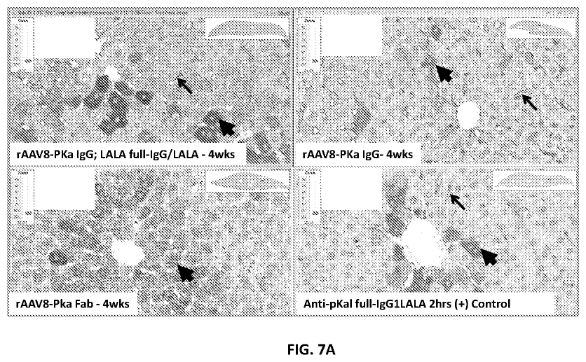

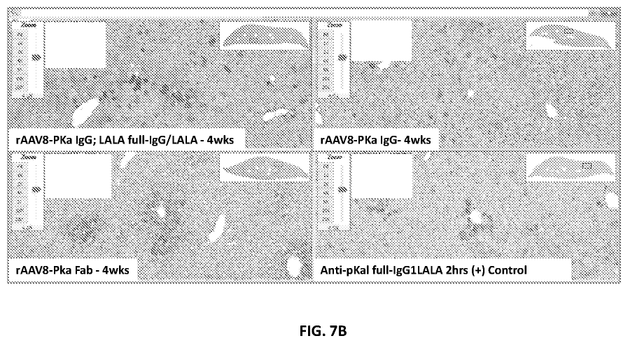

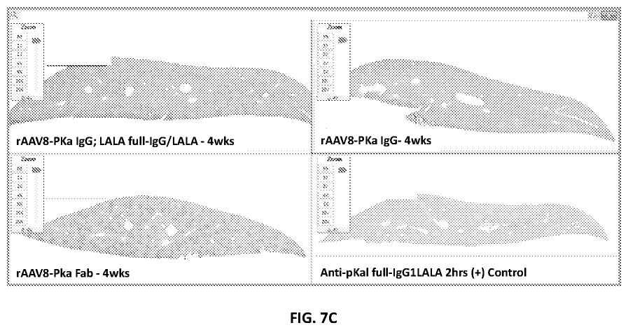

In this study, mice were injected with rAAV8/anti-PKa IgG or Fab and were euthanized after 4 weeks. Tissue specimen from livers were processed for immunohistochemistry (IHC). FIG. 6 shows positive immunostaining for anti-human IgG (anti-Fab domain detection) only in the mice that were injected with vector expressing anti-PKa-LALA vector (left image). Neither the empty vector injected mice tissue (middle) nor the uninjected mice exhibited immunostaining. FIGS. 7A-7C show high, medium and low magnification IHC images respectively of liver samples from mice injected with anti-PKa IgG+LALA, anti-PKa IgG and anti-PKa Fab vectors. Positive staining was observed in hepatocytes and liver sinusoidal cells as indicated by arrows.

### Example 5: Sustained Production of Anti-PKa Antibodies Following Administration of rAAV

Studies were performed to assess the percent activity of anti-PKa antibodies produced following administration of rAAV8 comprising anti-PKa IgG LALA heavy chain and light chain sequences. For these studies, mice were injected with either 1e10 vg and lell vg dose of rAAV anti-PKa IgG LALA vectors, followed by assessment for the presence of active anti-PKa IgG LALA after 2 weeks and after 4 weeks post administration of the rAAV vector. As a negative control for these studies, samples from non-immunized mice (time zero, “n/a” in FIG. 8) were assessed for active anti-PKA IgG LALA presence. As a positive control for these studies, samples from mice immunized with purified anti-PKa IgG LALA was assessed 2 hours post injection with purified anti-PKa IgG. The results from these studies is presented in FIG. 8.

The results show that administration of rAAV comprising anti-PKa IgG LALA heavy chain and light chain sequences resulted in sustained production of active anti-PKa IgG LALA throughout the assessed time points (2 weeks and 4 weeks). Surprisingly, the liver produced rAAV antibodies had an identical percent activity relative to the purified protein delivered by IV. This study demonstrates the feasibility of using rAAV comprising anti-PKa IgG LALA heavy chain and light chain sequences to obtain sustained expression of active anti-PKa IgG LALA following a single administration of the rAAV.

### Example 6: LC-MS Analysis of Intact and Processed Antibody

This study compares the mass spectra of two anti-PKa antibodies: 1) Purified or standard anti-PKa antibody, and 2) anti-PKa antibody produced in the rAAV8-treated mouse plasma sample. The mass spectra of both these antibodies were compared under following four conditions: 1) when both anti-PKa antibodies were intact, i.e., when the antibodies were in the mouse plasma; 2) when both anti-PKa antibodies were reduced using dithiothreitol (DTT); 3) when both anti-PKa antibodies were deglycosylated; and 4) when both anti-PKa antibodies were deglycosylated and reduced. To compare both anti-PKa antibodies in their intact forms, the purified anti-PKa antibody was added to a blank mouse plasma, and was then compared with anti-PKa antibody that was in the rAAV8-treated mouse plasma sample.

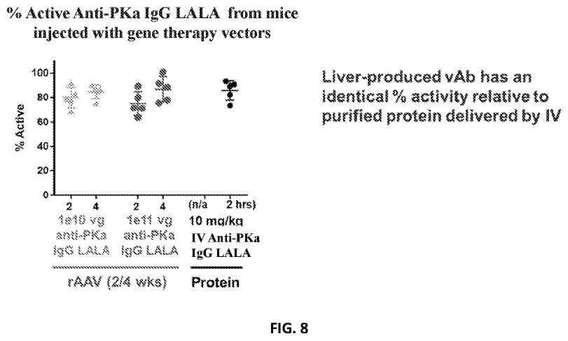

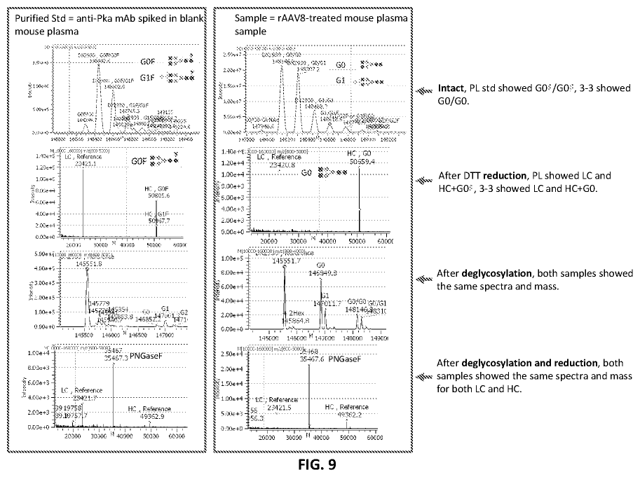

FIG. 9 depicts mass spectra of purified anti-PKa antibody and anti-PKa antibody produced in the rAAV8-treated mouse plasma under four different, as described above, conditions. Graphs at the left panel of the figure represent purified/standard antibody. Graphs at the right panel of the figure represent the anti-PKa antibody produced in the rAAV8-treated mouse plasma sample. As can be clearly seen, the molecular weight and the spectra of the purified anti-PKa antibody is identical to that of the anti-PKa antibody produced in the rAAV8-treated mouse plasma sample under similar deglycosylation conditions. For example, deglycosylated purified anti-PKa antibody (left panel, third graph from top) and deglycosylated anti-PKa antibody obtained from rAAV8-treated mouse plasma (right panel, third graph from top) both show same spectra and molecular weight. Deglycosylated and reduced purified anti-PKa antibody (left panel, bottom graph) and deglycosylated and reduced anti-PKa antibody obtained from rAAV8-treated mouse plasma (right panel, bottom graph) also show same spectra and mass for both light chain and heavy chain.

### Example 7: Evaluation of Ex Vivo Potency of Anti-PKa Antibody Produced in rAAV8-Treated Mouse Plasma

This study illustrates the ex vivo bioactivity of anti-PKa antibody produced in rAAV8-treated mouse plasma sample collected at 28 days after rAAV8 construct intravenous administration. In this study, the kallikrein-kinin pathway was activated in a control untreated mouse plasma sample by addition of ellagic acid in the presence of a gradual increase (titration) of an exogenous inhibitor, Takhzyro™. Takhzyro™ (lanadelumab-flyo) is an FDA approved fully human monoclonal antibody drug for preventing hereditary angioedema (HAE) attacks in 12 years or older patients. PKa activity in the plasma is monitored through the addition of a PKa-specific pro-fluorescent substrate (PFR-AMC) and subsequent fluorescent measurements made over time. To test the bioactivity of the anti-PKa antibody produced in the rAAV8-treated mouse plasma sample, the kallikrein-kinin pathway was similarly activated by addition of ellagic acid to plasma from these mice and PKa activity measured. Specifically, post-dose plasma from an individual rAAV8-treated mouse was serially diluted into a pre-dose plasma sample from the same mouse before the ellagic acid and PFR-AMC additions in order to measure a dose response.

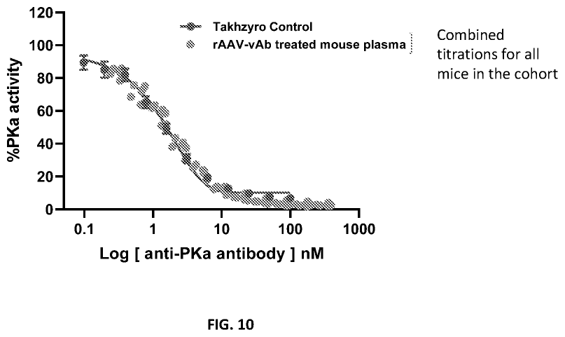

The bioactivity was measured in terms of percent inhibition of plasma kallikrein activity as a function of anti-PKa antibody concentration from these dilution series, where higher levels of antibody result in lower % PKa activity. FIG. 10 depicts the result of this study. The result demonstrates that the dose response of the anti-PKa antibody produced in the rAAV8-treated mouse is identical to the dose response of Takhzyro™, an FDA approved drug. This demonstrates that anti-PKa antibody produced in the rAAV8-treated mouse plasma has a very high integrity that is indistinguishable from Takhzyro™ drug product.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A recombinant adeno-associated viral (rAAV) vector encoding a full length antibody comprising an anti-plasma kallikrein antibody heavy chain and an anti-plasma kallikrein antibody light chain.

2. The rAAV vector of claim 1, wherein the anti-plasma kallikrein antibody heavy chain and the anti-plasma kallikrein antibody light chain are linked via a linker.

3. The rAAV vector of claim 2, wherein the linker comprises a cleavable linker.

4. The rAAV of claim 3, wherein the linker comprises a non-cleavable linker.

5. The rAAV vector of claim 1, wherein the anti-plasma kallikrein antibody heavy chain and the anti-plasma kallikrein antibody light chain are controlled by a single promoter.

6. The rAAV vector of claim 1, wherein the anti-plasma kallikrein antibody heavy chain and the anti-plasma kallikrein antibody light chain are controlled by separate promoters.

7. The rAAV vector of claims 5 or 6, wherein the single promoter or the separate promoter is selected from a ubiquitous promoter, a tissue-specific promoter, or a regulatable promoter.

8. The rAAV vector of claim 7, wherein the tissue-specific promoter is a liver-specific promoter.

9. The rAAV vector of claim 8, wherein the liver-specific promoter comprises a promoter selected from human transthyretin promoter (TTR), modified hTTR (hTTR mod.), α-Antitrypsin promoter, Liver Promoter 1 (LP1), TRM promoter, human factor IX pro/liver transcription factor-responsive oligomers, LSP, CMV/CBA promoter (1.1 kb), CAG promoter (1.7 kb), mTTR, modified mTTR, mTTR pro, mTTR enhancer, or the basic albumin promoter.

10. The rAAV vector of claim 9, wherein the liver-specific promoter is human transthyretin promoter (TTR).

11. The rAAV vector of claim 7, wherein the regulatable promoter is an inducible or repressible promoter.

12. The rAAV vector of claim 1, wherein the vector further comprises one or more of the following: a 5′ and a 3′ inverted terminal repeat, an intron upstream of the sequence, and a cis-acting regulatory module (CRM).

13. The rAAV vector of claim 1, wherein the vector further comprises a WPRE sequence.

14. The rAAV vector of claim 13, wherein the WPRE sequence is modified.

15. The rAAV vector of claim 14, wherein the WPRE contains a mut6delATG modification.

16. The rAAV vector of claim 12, wherein the CRM is liver-specific CRM.

17-48. (canceled)

49. A recombinant adeno-associated virus (rAAV) comprising an AAV8 capsid and an rAAV vector, said vector comprising:
a. a 5′ inverted terminal repeat (ITR);
b. a cis-acting regulatory module (CRM);
c. a liver specific promoter;
e. an anti-plasma kallikrein antibody heavy chain sequence and an anti-plasma kallikrein antibody light chain sequence;
f. a woodchuck hepatitis virus posttranscriptional regulatory element (WPRE); and
g. a 3′ ITR.

50-56. (canceled)

57. A method of treating a disease or disorder associated with a deficiency or dysregulation in the activated kallikrein-kinin pathway in a subject in need thereof comprising administering a recombinant adeno-associated viral vector (rAAV) of claim 1.

58. The method of claim 57, wherein the deficiency or dysregulation in the activated kallikrein-kinin pathway is a disease or disorder associated with a deficiency in C1 esterase inhibitor.

59-68. (canceled)
